(19) United States
(12) Patent Application Publication
(10) Pub. No.: US 2025/0353518 A1
(43) Pub. Date: Nov. 20, 2025

(54) INFORMATION PRESENTATION SYSTEM AND INFORMATION PRESENTATION METHOD

(71) Applicant: Panasonic Automotive Systems Co., Ltd., Kanagawa (JP)

(72) Inventor: Tetsuo MATSUSE, Osaka (JP)

(73) Assignee: Panasonic Automotive Systems Co., Ltd., Kanagawa (JP)

(21) Appl. No.: 19/205,118

(22) Filed: May 12, 2025

(30) Foreign Application Priority Data
May 20, 2024 (JP) 2024-082182

(51) Int. Cl. B60W 50/14; A61B 5/16; A61B 5/18

(52) U.S. Cl. CPC B60W 50/14; A61B 5/165; A61B 5/18; B60W 2555/20

(57) ABSTRACT

An information presentation system includes a first obtainer, a second obtainer, and a processing unit. The first obtainer obtains state anxiety information indicating state anxiety of an occupant of a vehicle. The second obtainer obtains trait anxiety information indicating trait anxiety of the occupant. The processing unit executes a process of presenting the occupant with a message for assisting the occupant in driving, based on the trait anxiety information, when the state anxiety information indicates that the state anxiety is occurring in the occupant.

### CROSS REFERENCE TO RELATED APPLICATION

The present application is based on and claims priority of Japanese Patent Application No. 2024-082182 filed on May 20, 2024.

### FIELD

The present disclosure relates to an information presentation system, an information presentation method, and a program for presenting information to, for example, an occupant of a vehicle.

### BACKGROUND

Patent Literature (PTL) 1 discloses a travel control system of a vehicle. This travel control system includes a travel control device that changes the vehicle's travel control so that when it is determined that the user is worried based on the results of the user's anxiety level measured by the user's anxiety level measuring device, the anxiety factor that is the source of the anxiety is determined based on the user's trait data with respect to the vehicle's travelling situation, and adjusts the degree of control of the vehicle's driving control based on the determined anxiety factor, thereby reducing the user's anxiety.

### CITATION LIST

### Patent Literature

PTL 1: Japanese Unexamined Patent Application Publication No. 2016-52881

### SUMMARY

However, the system according to PTL 1 can be improved upon.

In view of this, the present disclosure provides an information presentation system capable of improving upon the above related art.

An information presentation system according to one aspect of the present disclosure includes a first obtainer, a second obtainer, and a processing unit. The first obtainer obtains state anxiety information indicating state anxiety of an occupant of a vehicle. The second obtainer obtains trait anxiety information indicating trait anxiety of the occupant. The processing unit executes a process of presenting the occupant with a message for assisting the occupant in driving, based on the trait anxiety information, when the state anxiety information indicates that the state anxiety is occurring in the occupant. An information presentation method according to one aspect of the present disclosure includes: obtaining state anxiety information indicating state anxiety of an occupant of a vehicle; obtaining trait anxiety information indicating trait anxiety of the occupant; and executing a process of presenting the occupant with a message for assisting the occupant in driving, based on the trait anxiety information, when the state anxiety information indicates that the state anxiety is occurring in the occupant.

A program according to one aspect of the present disclosure causes one or more processors to execute the information presentation method.

The information presentation system and the like of the present disclosure is capable of improving upon the above related art.

### BRIEF DESCRIPTION OF DRAWINGS

These and other advantages and features of the present disclosure will become apparent from the following description thereof taken in conjunction with the accompanying drawings that illustrate a specific embodiment of the present disclosure.

FIG. 1 is an explanatory diagram of an example of information presented by an information presentation system according to an embodiment.

FIG. 2 is a block diagram showing an outline of the information presentation system according to the embodiment.

FIG. 3 is an explanatory diagram of an example of obtaining state anxiety information by the first obtainer of the information presentation system according to the embodiment.

FIG. 4 is an explanatory diagram of an example of obtaining situation information by the third obtainer of the information presentation system according to the embodiment.

FIG. 5 is a flow chart showing an example of the operation of the information presentation system according to the embodiment.

FIG. 6 is a diagram showing an example of a first message to be presented to an occupant in the information presentation system according to the embodiment.

FIG. 7 is a diagram showing an example of a second message to be presented to an occupant in the information presentation system according to the embodiment.

FIG. 8 is a diagram showing an example of a third message to be presented to an occupant in the information presentation system according to the embodiment.

FIG. 9 is a diagram showing an example of a fourth message to be presented to an occupant in the information presentation system according to the embodiment.

FIG. 10 is a diagram showing an example of a fifth message to be presented to an occupant in the information presentation system according to the embodiment.

FIG. 11 is a diagram showing an example of a message to be presented to an occupant in the information presentation system according to a variation of the embodiment.

### DESCRIPTION OF EMBODIMENT

Hereinafter, an embodiment will be described in detail with reference to the drawings.

It should be noted that the embodiment explained below shows a comprehensive or specific example. The numerical values, shapes, materials, components, installation positions and connection forms of the components, steps, order of steps, and the like shown in the following embodiment are merely examples and are not intended to limit the present disclosure.

In addition, among the components in the following embodiments, the components not described in the independent claims are described as arbitrary components.

In addition, each figure is a schematic view and is not necessarily exactly illustrated. In addition, in each figure, the same components are denoted by the same reference numerals.

### EMBODIMENT

### <Outline>

First, the points that the inventor focuses on will be explained.

Conventionally, as a technology for supporting the driving of a vehicle by an occupant, a technology for detecting a state of anxiety (so-called state anxiety) of an occupant of a vehicle while driving, and providing the occupant with a support according to the detected state of anxiety, has been disclosed, for example, in PTL 1. However, the technology disclosed in PTL 1 takes into consideration the state of anxiety (state anxiety) of the occupant detected during driving, but does not take into consideration the trait of the occupant's ease of becoming an anxious state (so-called trait anxiety), and there is a problem that it is not possible to provide effective support to the occupant.

Here, state anxiety and trait anxiety are concepts proposed by Spielberger et al. in 1966. State anxiety refers to anxiety as a temporary emotional state that a person has about a specific point in time, scene, event, or object. On the other hand, trait anxiety refers to the tendency to be anxious due to a person's personality and the like (Reference: Koga, “State Anxiety and Trait Anxiety”, Japanese Psychological Review, 1980, Vol. 23, No. 3).

In general, people with high trait anxiety tend to respond to a relatively wide range of situations as dangerous or threat, and are more likely to show elevated state anxiety than people with low trait anxiety. That is, for occupants with high trait anxiety, support according to the state anxiety detected during driving may not be sufficient, or it may not be possible to dispel the anxiety.

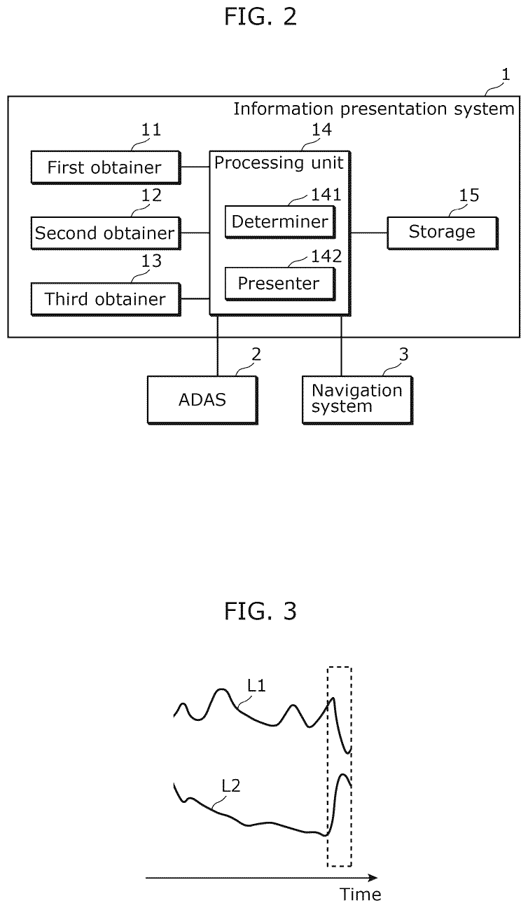

Therefore, in view of the above points, the inventor has found that the assistance from a psychological perspective is effective for people with high trait anxiety by presenting information to the occupant using information presentation system 1 (see FIG. 2) according to the embodiment. This point will be explained in detail below.

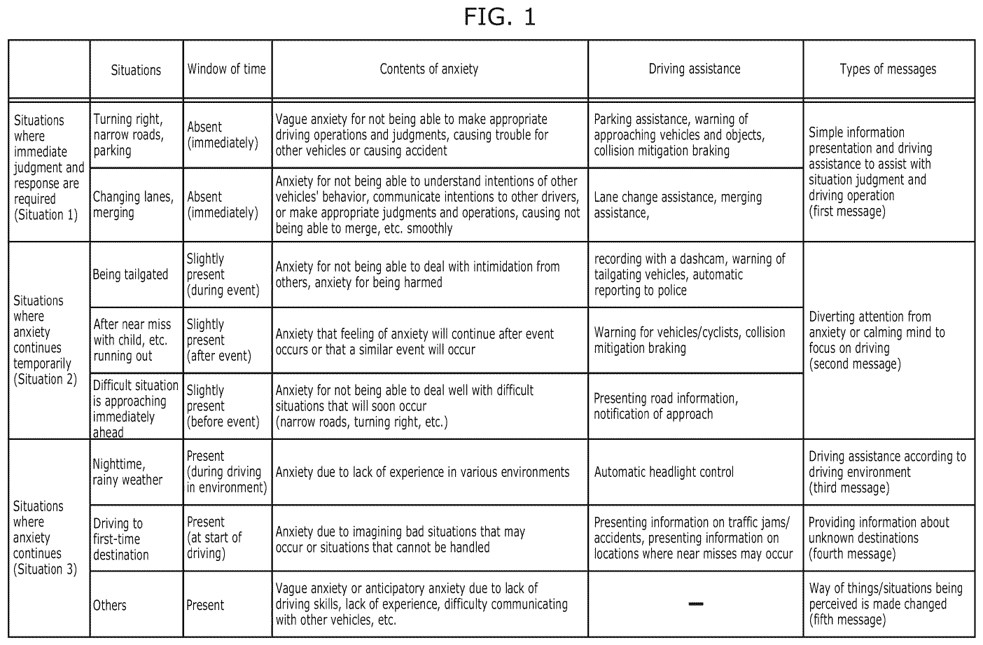

FIG. 1 is an explanatory diagram of an example of information presented by information presentation system 1 according to the embodiment. As shown in FIG. 1, situations in which the occupant feels anxious are roughly divided into the first, second and third situations.

The first situation is a situation in which immediate judgment and immediate response are required. In other words, the first situation is a situation in which state anxiety is occurring in the occupant and there is no (or almost no) window of time for the occupant to cope with the situation. The first situation is a situation that can occur when a vehicle turns right, when a vehicle is traveling on a narrow road, when it is attempted to park a vehicle, when a vehicle is changing lanes, or when a vehicle merges, for example, as shown in the “Situation” column in FIG. 1.

The second situation is a situation in which anxiety continues temporarily. In other words, the second situation is a situation in which state anxiety is occurring in the occupant and there is a window of time (for example, about 10 seconds, a few minutes, or the like). The second situation is, for example, as shown in the “Situation” column in FIG. 1, when an occupant is being tailgated, a situation that can occur after a so-called near miss caused by a child or the like running out, or a situation in which a difficult situation (a narrow road where it is difficult to pass each other, or a right turn, or the like) is approaching immediately ahead.

The third situation is a situation in which anxiety continues. In other words, the third situation is a situation in which state anxiety is occurring in the occupant and the window of time is longer than a predetermined time. The third situation is, for example, as shown in the “Situation” column in FIG. 1, a situation in which the vehicle is in an environment that affects the occupant's field of view, such as at nighttime or in the rainy weather, or a situation in which the destination, such as a road or an end of journey, is the first time for the occupant to go to.

Here, existing systems mounted on a vehicle such as advanced drive assistant system (ADAS) 2 (see FIG. 2), navigation system 3 (see FIG. 2), or the like assist the occupant in driving according to the situation in which the occupant is placed.

For example, when the occupant is in a first situation, as shown in the “Driving assistance” column in FIG. 1, the existing system assists the occupant in driving by performing functions such as parking assistance, warning of approach of vehicles or objects, collision mitigation braking, and lane change assistance. In addition, it is also expected that in the future, it will assist the occupant in driving by executing the function of merging assistance.

In addition, for example, when the occupant is in a second situation, as shown in the “Driving assistance” column in FIG. 1, the existing system assists the occupant in driving by performing the function of recording the drive recorder. In addition, it is also expected that in the future, the occupant will be assisted in driving by performing the function of warning to tailgating vehicles or automatic reporting to police.

In addition, for example, when the occupant is in a third situation, especially when the vehicle is in an environment in which the vehicle affects the occupant's field of view, as shown in the “Driving assistance” column in FIG. 1, the existing system assists the occupant in driving by performing functions such as automatic headlight control. In addition, for example, when the occupant is in a third situation, especially when the destination is the first time for the occupant to go to, as shown in the “Driving assistance” column in FIG. 1, the existing system assists the occupant in driving by performing functions such as presenting information on traffic jam or accidents, or presenting information on the location where near misses occurred.

Driving assistance for occupants using the existing system mentioned above is an assistance according to state anxiety, and is considered effective for occupants with low trait anxiety. However, for occupants with high trait anxiety, it is considered that it would be difficult to resolve their state anxiety by using driving assistance using existing systems alone.

Therefore, information presentation system 1 according to the embodiment assists the occupant from a psychological perspective and aims to eliminate state anxiety by presenting the occupant with a message according to the situation in which the occupant is placed.

For example, when the occupant is in the first situation, as shown in the “Type of message” column in FIG. 1, information presentation system 1 presents the occupant with a simple information presentation for assisting the occupant in the situation judgment or driving operation, or a message for providing driving assistance. Such a message may be referred to as the “first message” below.

In addition, for example, when the occupant is in a second situation, as shown in the “Type of message” column in FIG. 1, information presentation system 1 presents the occupant with the message for diverting attention of the occupant from anxiety or the message for calming an anxious feeling and focusing on driving. Such a message may be referred to as the “second message” below.

In addition, for example, when the occupant is in a third situation, especially when the occupant is in an environment that affects the occupant's field of view, as shown in the “Type of message” column in FIG. 1, information presentation system 1 presents the occupant with a message for providing driving assistance according to the travel environment. Such a message may be referred to as the “third message” below.

In addition, for example, when the occupant is in a third situation, especially when the destination is the first time for the occupant to go to, as shown in the “Type of message” column in FIG. 1, information presentation system 1 presents the occupant with a message for providing information on an unknown destination. Such a message may be referred to as the “fourth message” below.

In addition, for example, when the occupant is in a third situation other than the above, as shown in the “Type of message” column in FIG. 1, information presentation system 1 presents the occupant with a message for causing the occupant to change the way things or situations are perceived, in other words, a message for promoting “cognitive change (reevaluation)” (hereinafter also referred to as “reevaluation in cognitive change”). Such a message may be referred to as the “fifth message” below.

Here, “cognitive change (reevaluation)” is a method that belongs to cognitive change among situation selection, situation correction, attention allocation, cognitive change, and response adjustment, known as methods for controlling emotions, and refers to changing the evaluation of a situation or one's abilities in order to change the emotions evoked by the situation. It should be noted that for more information on the methods for controlling the emotions mentioned above, see, for example, “Handbook of Emotion Regulation, Chapter 12, et al., 2022”, and “J. J. Gross, Antecedent- and response-focused emotions regulation: Divergent consequences for experience, expression, and physiology, Journal of Personality and Social Psychology, 1998”.

### <Configuration>

Next, the configuration of information presentation system 1 according to the embodiment will be described. FIG. 2 is a block diagram showing an outline of information presentation system 1 according to the embodiment. Information presentation system 1 is used for mobile bodies such as vehicles or the like, and is a system for assisting in driving the vehicle.

In the embodiment, information presentation system 1 is realized by on-vehicle devices, but may be realized by external devices brought in from outside the vehicle. In addition, in the embodiment, the occupant targeted by information presentation system 1 is a driver seated in the driver's seat of the vehicle, but it may be an occupant other than the driver seated in the passenger seat or the rear seat.

Information presentation system 1 is realized, for example, by an electronic control unit (ECU). As shown in FIG. 2, information presentation system 1 includes first obtainer 11, second obtainer 12, third obtainer 13, processing unit 14, and storage 15. It should be noted that storage 15 may not be included as a component of information presentation system 1.

In addition, information presentation system 1 is connected to ADAS 2 and navigation system 3, and when ADAS 2 and navigation system 3 perform the driving assistance function, it links with ADAS 2 and navigation system 3 to present a message to the occupant. It should be noted that information presentation system 1 may present a message to the occupant without linking with ADAS 2 and navigation system 3.

First obtainer 11 obtains state anxiety information indicating the state anxiety of the occupant of the vehicle. In the embodiment, first obtainer 11 obtains the biological information of the occupant, and detects the state anxiety based on the obtained biological information, thereby obtaining the state anxiety information. Here, the biological information may include, for example, the heartbeat of the occupant, the electro dermal activity (EDA) of the occupant, or the like. It should be noted that for information on detecting state anxiety based on biological information, see, for example, “Koba and Ohsuga, “Estimation of Anxiety Level using Physiological Measures to VR Exposure Therapy System”, Transactions of Japanese Society for Medical and Biological Engineering, 2020″.

Specifically, first obtainer 11 successively obtains information indicating the heartbeat of the occupant and information indicating the EDA from, for example, electrodes mounted on the steering wheel. It should be noted that the occupant's heartbeat and EDA may be obtained from a wearable device such as a smart watch or the like, which is attached to the occupant's wrist, arm, head, or the like. As an example of an algorithm for detecting a state anxiety state, first obtainer 11 obtains state anxiety information indicating that state anxiety is occurring in the occupant when the average value of the R-R interval (RRI), which is the variation time series data of the obtained heartbeat, decreases, and the average value of the skin conductance (SC), which is information on the obtained EDA, rises significantly. It should be noted that in the following, “a state anxiety is occurring in the occupant” may be read as “a state anxiety occurring in the occupant is in a relatively high state.”

FIG. 3 is an explanatory diagram of an example of obtaining state anxiety information by first obtainer 11 of information presentation system 1 according to the embodiment. In FIG. 3, solid line L1 represents a time series change in the average value of the occupant's RRI, and solid line L2 represents a time series change in the average value of the SC of EDA. During the time surrounded by the dashed line in FIG. 3, the average RRI value decreases, and the average EDA SC value increases significantly. For this reason, in the example shown in FIG. 3, first obtainer 11 obtains state anxiety information indicating that state anxiety is occurring in the occupant during the time surrounded by a dashed line.

Second obtainer 12 obtains trait anxiety information indicating the trait anxiety of the occupant. Hereinafter, examples of obtaining trait anxiety information by second obtainer 12 are listed. In the first and second obtaining examples shown below, second obtainer 12 obtains trait anxiety information based on the results of the answers to the questions regarding trait anxiety for the occupant. In addition, in the third and fourth obtaining examples shown below, second obtainer 12 obtains trait anxiety information based on the degree of occurrence of state anxiety during driving of the occupant.

In the first obtaining example, for example, questions are asked to the occupant when the vehicle is delivered, or questions are asked to the occupant through an on-board display when the vehicle is used for the first time. There are three questions: (1) whether you are prone to anxiety while driving, (2) how much you have a driving history, and (3) whether you are not good at driving. Regarding (1), if you answer “I'm always anxious,” then the score is 3 points, if you answer “I'm often anxious,” then the score is 1 point, and if you answer “I'm sometimes anxious/I'm hardly anxious,” then the score is 0 point. In addition, regarding (2), if you answer your driving history is “less than one year”, then the score is 2 points, if you answer your driving history is “less than three years”, then the score is 1 point, and if you answer your driving history is “more than three years”, then the score is 0 point. In addition, regarding (3), if you answer you are “poor at driving”, then the score is 2 points, if you answer you are “not very good at driving”, then the score is 1 point, and if you answer you are “neither good nor poor at driving, rather good at driving, or good at driving”, then the score is 0 point.

Then, when the occupant is asked only about (1), second obtainer 12 obtains trait anxiety information indicating that the trait anxiety is “high” in the case of 3 points, obtains trait anxiety information indicating that the trait anxiety is “medium” in the case of 1 point, and obtains trait anxiety information indicating that the trait anxiety is “low” in the case of 0 point. In addition, when the occupant is asked about (1) and (2), second obtainer 12 obtains trait anxiety information indicating that the trait anxiety is “high” when the total points are 4 to 5 points, obtains trait anxiety information indicating that the trait anxiety is “medium” when the total points are 2 to 3 points, and obtains trait anxiety information indicating that the trait anxiety is “low” when the total points are 0 to 1 point. In addition, when the occupant is asked about (1), (2), and (3), second obtainer 12 obtains trait anxiety information indicating that the trait anxiety is “high” when the total points are 6 to 7 points, obtains trait anxiety information indicating that the trait anxiety is “medium” when the total points are 3 to 5 points, and obtains trait anxiety information indicating that the trait anxiety is “low” when the total points are 0 to 2 points.

In the first obtaining example, trait anxiety information is obtained by asking questions to the occupant based on the basis shown below. That is, a survey was conducted on dozens of people, including people who are poor at driving, people who are good at driving, and people who are neither good nor poor at driving, to determine whether they feel anxious while driving, and how often they feel anxious if they feel anxious. As a result, the correlation was obtained: the better the driver was at driving, the less anxious the driver felt (that is, the lower the trait anxiety), and the worse the driver was at driving, the more anxious the driver felt (that is, the higher the trait anxiety).

In addition, a survey was conducted on dozens of people, including people with no driving history, short driving history, and long driving history, to determine whether they feel anxious while driving, and how often they feel anxious if they feel anxious. As a result, the longer the driving history, the less anxiety is felt (that is, the lower the trait anxiety), and the shorter the driving history, the more anxiety is felt (that is, the higher the trait anxiety).

In the second obtaining example, second obtainer 12, for example, performs state-trait anxiety inventory (STAI) to the occupant when the vehicle is delivered, or performs STAI to the occupant through an on-board display when the vehicle is used for the first time. The STAI is an examination that asks occupant questions about trait anxiety. For information on STAI, see “STAI, Spielberger, 1970”, “New edition STAI (Japanese version), https://www.jitsumu-kyouzai.com/wellness/show_product.php?pid=75”.

Then, second obtainer 12 obtains trait anxiety information indicating that the trait anxiety is “high” when the STAI response result is “5” out of five levels, obtains trait anxiety information indicating that the trait anxiety is “medium” when the STAI response result is “4 or 3”, and obtains trait anxiety information indicating that the trait anxiety is “low” when the STAI response result is “2 or 1”.

In the third obtaining example, second obtainer 12 refers to the history of the occupant's state anxiety information obtained by first obtainer 11 when the vehicle is travelling. Specifically, second obtainer 12 calculates the ratio of the distance (or time) of a section in which state anxiety is occurring in the occupant with respect to the travel distance (or travel time) based on the history of the vehicle traveling for a certain time, and obtains trait anxiety information from the ratio. For example, second obtainer 12 obtains trait anxiety information indicating that the trait anxiety is “high” when the ratio is 10% or more, obtains trait anxiety information indicating that the trait anxiety is “medium” when the ratio is 5% or more and less than 10%, and obtains trait anxiety information indicating that the trait anxiety is “low” when the ratio is less than 5%.

In the fourth obtaining example, second obtainer 12 refers to the history of the occupant's state anxiety information obtained by first obtainer 11 when the vehicle is traveling, and the history of the situation information, obtained by third obtainer 13 (to be described later), on the situation in which the occupant is placed when the vehicle is travelling. Specifically, second obtainer 12 calculates the ratio of a state anxiety that occurs in the occupant in a specific situation based on the history of a vehicle travelling for a certain time, and obtains trait anxiety information from the ratio. Here, the specific situation is a situation in which state anxiety is likely to occur in an occupant with high trait anxiety. The specific situation is the first, second or third situation already mentioned.

For example, second obtainer 12 obtains trait anxiety information indicating that the trait anxiety is “high” when the ratio is 30% or more, obtains trait anxiety information indicating that the trait anxiety is “medium” when the ratio is 10% or more and less than 30%, and obtains trait anxiety information indicating that the trait anxiety is “low” when the ratio is less than 10%.

Third obtainer 13 obtains situation information on the situation in which the occupant is placed. In the embodiment, third obtainer 13 obtains situation information based on information from various devices mounted on the vehicle, information from ADAS 2, information from navigation system 3, and the like.

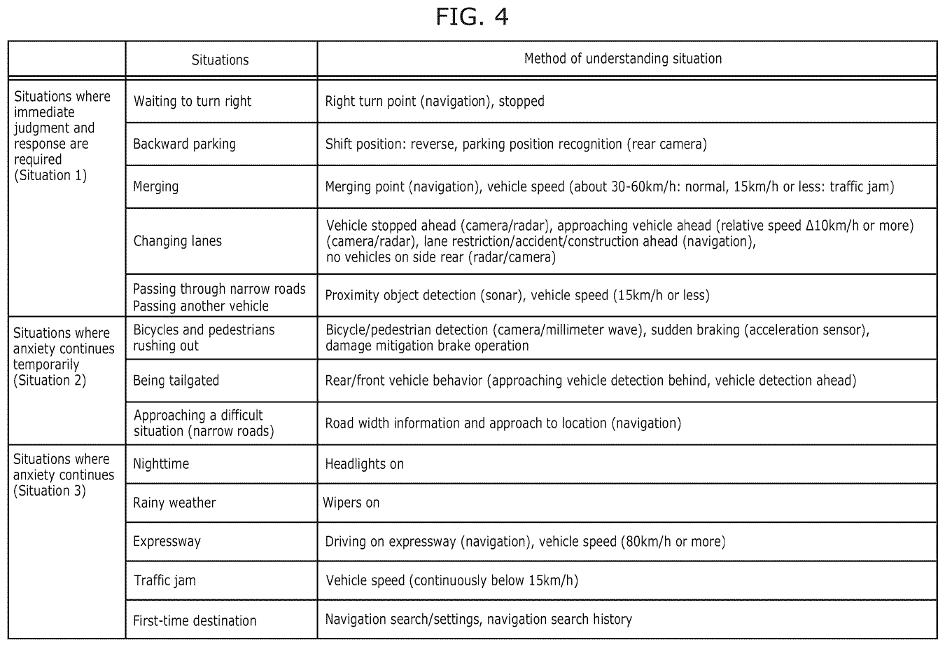

FIG. 4 is an explanatory diagram of an example of obtaining situation information by third obtainer 13 of information presentation system 1 according to the embodiment.

First, an example of the case in which third obtainer 13 obtains situation information indicating that the situation in which the occupant is placed is the first situation (that is, a situation in which immediate judgment and immediate response are required) will be described. For example, third obtainer 13 obtains situation information indicating that the vehicle is waiting to turn right, when navigation system 3 indicates that the vehicle's current position is at a right turn point and the vehicle is stopped. In addition, for example, third obtainer 13 obtains situation information indicating that the vehicle is during a backward parking, when the shift lever is in a retracted position and the rear camera recognizes the parking position. In addition, for example, third obtainer 13 obtains situation information indicating that the vehicle is in the process of merging, when navigation system 3 indicates that the vehicle's current position is at a merging point and the vehicle's speed is about 30 to 60 km/h (or 15 km/h or less if the vehicle is in a traffic jam).

In addition, for example, when a camera or radar indicates that there is a stopped vehicle in front of the vehicle, a camera or radar indicates that the vehicle is approaching a vehicle in front, or navigation system 3 indicates that the vehicle's current position is in a lane restriction, accident, or construction area, and the camera or radar indicates that there are no other vehicles to the side or rear of the vehicle, third obtainer 13 obtains situation information indicating that the vehicle is in a situation in which the vehicle is changing lanes. In addition, for example, third obtainer 13 obtains situation information indicating that the vehicle is passing through a narrow road or that the vehicle is passing another vehicle when the sonar detects an object approaching the vehicle and the vehicle's speed is 15 km/h or less.

Next, an example of the case in which third obtainer 13 obtains situation information indicating that the situation in which the occupant is placed is a second situation (that is, a situation in which anxiety is temporarily continued). For example, third obtainer 13 obtains situation information indicating that a bicycle or pedestrian has run out in front of the vehicle when the camera or millimeter wave sensor detects a bicycle or pedestrian, and when the acceleration sensor detects a sudden braking or ADAS 2 performs a damage mitigation braking operation. In addition, for example, third obtainer 13 obtains situation information indicating that the vehicle is being tailgated when the approach of a rear vehicle or a front vehicle to the vehicle is repeatedly detected in a short time, or when the extremely close proximity continues for a certain time.

Next, an example of the case in which third obtainer 13 obtains situation information indicating that the situation in which the occupant is placed is a third situation (that is, a situation where anxiety continues) will be described. For example, third obtainer 13 obtains situation information indicating that the vehicle is travelling at nighttime when the headlights are lit. In addition, for example, when the wiper is in operation, third obtainer 13 obtains situation information indicating that the vehicle is travelling in the rainy weather. In addition, for example, when navigation system 3 indicates that the vehicle is currently located on a highway and the vehicle's speed is 80 km/h or more, third obtainer 13 obtains situation information indicating that the vehicle is travelling on a highway. In addition, for example, third obtainer 13 obtains situation information indicating that the occupant is heading to a destination that is new to the occupant, based on the search results, settings, or search history of navigation system 3.

Processing unit 14 executes various processes for assisting the occupant of the vehicle in driving. In the embodiment, processing unit 14 includes determiner 141 and presenter 142. Processing unit 14 executes, for example, a program stored in the internal memory in advance, thereby performing the functions of determiner 141 and presenter 142.

When the state anxiety information indicates that state anxiety is occurring in the occupant, determiner 141 executes a process for determining whether to present a message to the occupant based on the trait anxiety information. In the embodiment, determiner 141 determines to present a message to the occupant when the value of the trait anxiety in the trait anxiety information is higher than the threshold value, and determines that the message is not presented to the occupant when the value of the trait anxiety is lower than the threshold value. Specifically, when determiner 141 has obtained trait anxiety information indicating that the trait anxiety is “high”, it determines to present a message to the occupant. On the other hand, when determiner 141 has obtained trait anxiety information indicating that the trait anxiety is “medium” or “low”, it determines not to present a message to the occupant.

In addition, when determiner 141 determines to present a message to the occupant, it executes a process of determining the type of message according to the situation information. The process for determining the type of message by determiner 141 will be described in detail in the <Operation> described later.

Presenter 142 executes a process of presenting the message determined by determiner 141 to the occupant. In the embodiment, presenter 142 reads out a message to be presented from a plurality of candidate messages stored in storage 15, and then outputs the read-out message as a voice from the speaker, thereby presenting the message to the occupant. The voice may be a machine voice or a recorded voice uttered by a person.

It should be noted that presenter 142 may generate a message to be presented using, for example, a message generation model that has been machine-learned in advance so as to output a message using situation information as input. In this case, it is not necessary to store a plurality of message candidates in storage 15. In addition, in this case, no process by determiner 141 to determine the type of message is necessary.

Storage 15 is, for example, a semiconductor memory, and is a dedicated or general purpose memory for storing information used in information presentation system 1. Storage 15 stores, for example, a plurality of candidates for messages that can be presented to the occupant by presenter 142.

### <Operation>

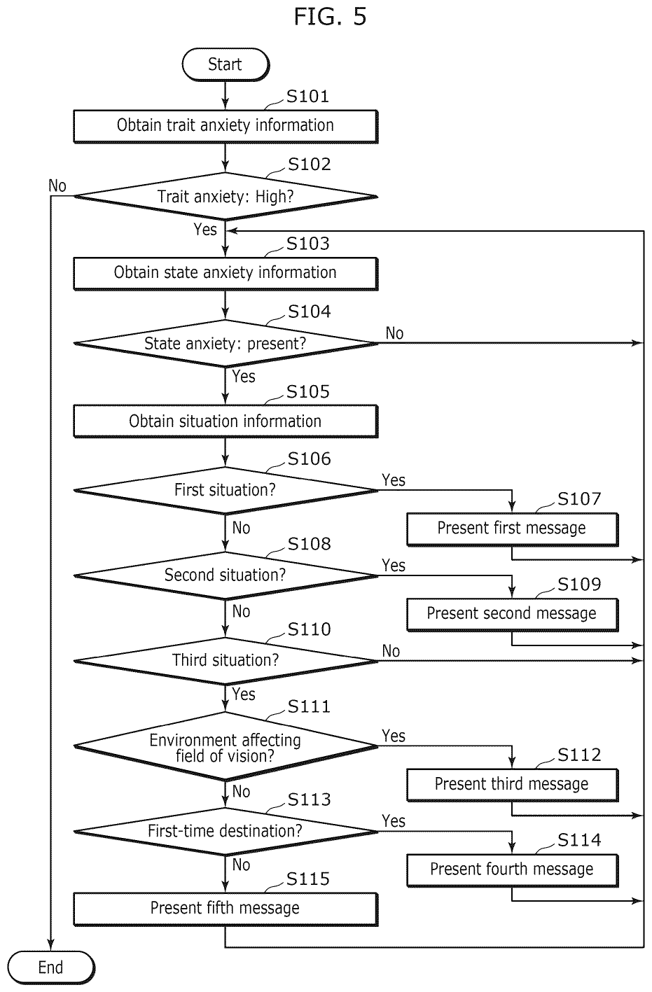

Hereinafter, the operation of information presentation system 1 according to the embodiment will be described with reference to FIG. 5. FIG. 5 is a flow chart showing an example of the operation of information presentation system 1 according to the embodiment.

First, second obtainer 12 obtains trait anxiety information (S101). Step S101 is executed, for example, when the vehicle is started up. It should be noted that when the trait anxiety information has already been obtained and stored in storage 15, second obtainer 12 obtains the trait anxiety information by reading out the trait anxiety information from storage 15. When the trait anxiety information indicates that the trait anxiety is “high” (S102: Yes), determiner 141 determines to present a message to the occupant while the vehicle is travelling (including temporary stop of the vehicle). On the other hand, when the trait anxiety information indicates that the trait anxiety is “medium” or “low” (S102: No), determiner 141 determines not to present a message to the occupant while the vehicle is travelling.

Next, first obtainer 11 sequentially obtains the state anxiety information while the vehicle is traveling (S103). While the state anxiety information indicates that no state anxiety is occurring in the occupant (S104: No), step S103 is repeated. Then, if the state anxiety information indicates that state anxiety is occurring in the occupant (S104: Yes), third obtainer 13 obtains the situation information (S105). It should be noted that step S105 may be executed sequentially without relying on step S104.

Next, determiner 141 determines the type of message to be presented to the occupant according to the situation information. It should be noted that when situation information indicating that the situation does not fall under any of the first, second, or third situations is obtained (S106: No, S108: No, S110: No), determiner 141 does not determine the type of message. In this case, information presentation system 1 executes step S103 again.

If third obtainer 13 obtains situation information indicating that the situation in which the occupant is placed is the first situation (that is, a situation in which immediate judgment and immediate response are required) (S106: Yes), determiner 141 determines to present the first message to the occupant. Then, presenter 142 presents the first message to the occupant (S107). After executing step S107, information presentation system 1 executes step S103 again.

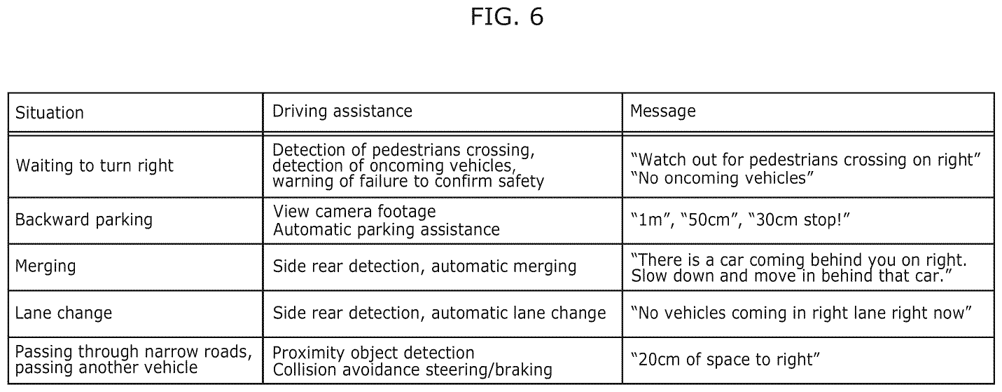

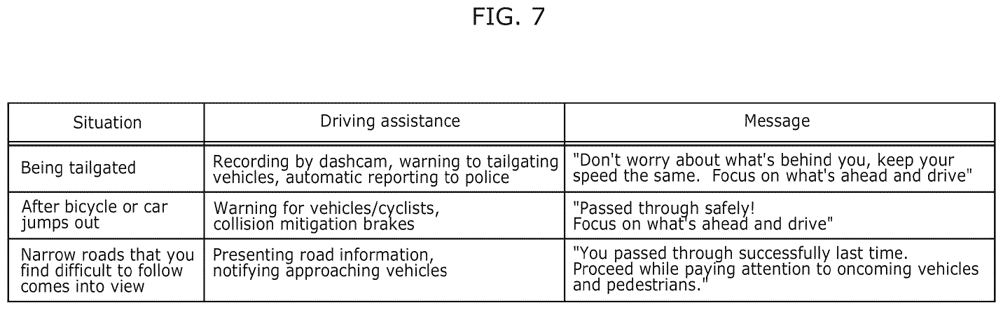

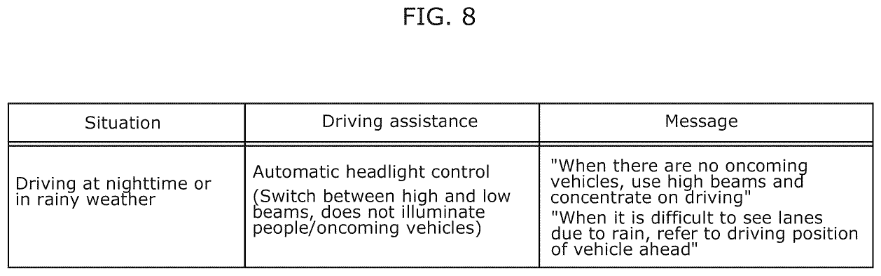

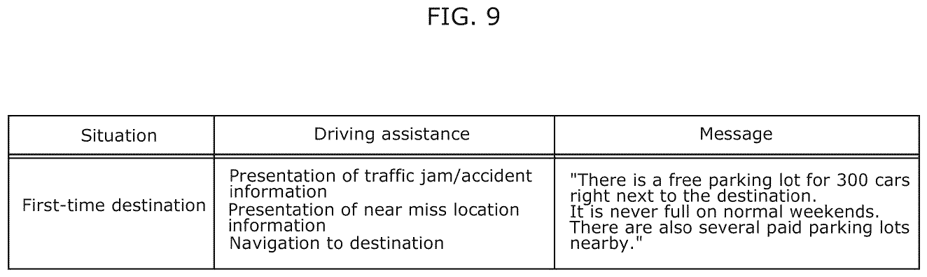

FIG. 6 is a diagram showing an example of a first message to be presented to an occupant in information presentation system 1 according to the embodiment. As shown in the “Message” column in FIG. 6, when the situation in which the occupant is placed is the first situation, presenter 142 presents the occupant with a simple information presentation for assisting the occupant in the situation judgment or driving operation, or a message for providing driving assistance. It should be noted that the “Driving assistance” column in FIG. 6 shows the contents of driving assistance executed by various devices mounted on the vehicle, ADAS 2, or navigation system 3 when presenter 142 presents the first message. The same applies to each of the “Driving assistance” columns shown in FIG. 7, FIG. 8 and FIG. 9, which will be explained below.

Returning to FIG. 5, when third obtainer 13 obtains situation information indicating that the situation in which the occupant is placed is a second situation (that is, a situation in which anxiety is temporarily continued) (S106: No, S108: Yes), determiner 141 determines to present the second message to the occupant. Then, presenter 142 presents the second message to the occupant (S109). After executing step S109, information presentation system 1 executes step S103 again.

FIG. 7 is a diagram showing an example of a second message to be presented to an occupant in information presentation system 1 according to the embodiment. As shown in the “Message” column in FIG. 7, when the situation in which the occupant is placed is the second situation, presenter 142 presents the occupant with the message for diverting attention of the occupant from the anxiety and focuses on driving, or the message for calming an anxious feeling.

Returning to FIG. 5, when third obtainer 13 obtains situation information indicating that the situation in which the occupant is placed is the third situation (that is, the situation in which anxiety continues) (S108: No, S110: Yes), determiner 141 further determines the type of message to be presented to the occupant according to the contents of the third situation.

Specifically, when the environment affects the occupant's field of view (S111: Yes), determiner 141 determines to present a third message to the occupant. Then, presenter 142 presents the third message to the occupant (S112). After executing step S112, information presentation system 1 executes step S103 again.

FIG. 8 is a diagram showing an example of a third message to be presented to the occupant in information presentation system 1 according to the embodiment. As shown in the “Message” column in FIG. 8, when the situation in which the occupant is placed is the third situation and the environment affects the occupant's field of view, presenter 142 presents the occupant with a message to provide driving assistance according to the travel environment.

Returning to FIG. 5, when the destination is the first time for the occupant (S111: No, S113: Yes), determiner 141 determines to present a fourth message to the occupant. Then, presenter 142 presents the fourth message to the occupant (S114). After executing step S114, information presentation system 1 executes step S103 again.

FIG. 9 is a diagram showing an example of a fourth message to be presented to an occupant in information presentation system 1 according to the embodiment. As shown in the “Message” column in FIG. 9, presenter 142 presents the occupant with the message to provide information on an unknown destination when the situation in which the occupant is placed is the third situation and the destination is the first time for the occupant.

Returning to FIG. 5, when the third situation is a situation other than the above (S113: No), determiner 141 determines to present a fifth message to the occupant. Then, presenter 142 presents the fifth message to the occupant (S115). After executing step S115, information presentation system 1 executes step S103 again.

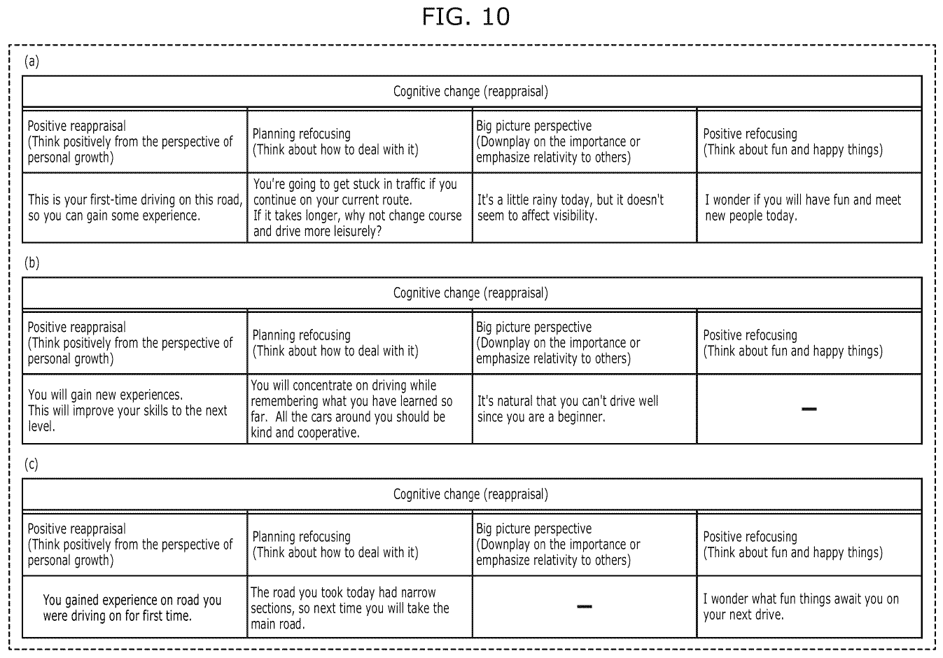

FIG. 10 is a diagram showing an example of the fifth message to be presented to the occupant in information presentation system 1 according to the embodiment. (a) in FIG. shows an example of the fifth message to be presented to the occupant when the driving begins, (b) in FIG. 10 shows an example of the fifth message to be presented to the occupant during driving, and (c) in FIG. 10 shows an example of the fifth message to be presented to the occupant when the driving ends. As shown in FIG. 10, presenter 142 presents the occupant with a message for causing the occupant to change the way things or situations are perceived, that is, for promoting “cognitive change (reevaluation).”

In the embodiment, as shown in FIG. 10, the message presented by presenter 142 includes at least one of a message that promotes positive reevaluation, which is a type of subcategorization of “cognitive change (reevaluation)”, a message that promotes refocus on the plan, a message that promotes a big picture perspective, or a message that promotes positive refocusing. Positive reevaluation refers to thinking positively about the current situation from the perspective of personal growth. Refocusing on the plan refers to thinking about how coping with the current situation can change it. A big picture perspective refers to underestimating the severity of the current situation, or thinking of the current situation as better than other negative situations. Positive refocusing refers to thinking about positive things that have nothing to do with the current situation.

### Advantages

The advantages of information presentation system 1 according to the embodiment will be explained below. As mentioned above, when state anxiety is occurring in an occupant, information presentation system 1 according to the embodiment executes a process of presenting the occupant with a message for assisting the occupant in driving, based on the trait anxiety information indicating the trait anxiety of the occupant. For this reason, information presentation system 1 according to the embodiment has the advantage that it is possible to assist the occupant from a psychological perspective, taking trait anxiety into account, and that it is easier to reduce the anxiety that the occupant of the vehicle has when driving. In particular, information presentation system 1 according to the embodiment has the advantage that even if the anxiety of the occupant cannot be dispelled only by the support according to the state anxiety of the occupant detected during driving by, for example, ADAS 2 or the like, it is easier to dispel the anxiety by presenting a message to the occupant.

### VARIATIONS

The information presentation system according to the present disclosure has been described above based on the above-mentioned embodiment, but the present disclosure is not limited to the embodiment. Forms obtained by applying various modifications to the embodiment conceived by a person skilled in the art without departing from the spirit of the present disclosure may also be included in the scope of this disclosure.

In the above embodiment, processing unit 14 only presents a message to the occupant when the value of the trait anxiety is higher than the threshold value (in other words, the trait anxiety is relatively high), but the present disclosure is not limited thereto. For example, processing unit 14 (determiner 141) may determine the type of message to be presented to the occupant according to the high and low values indicating trait anxiety (in other words, the degree of trait anxiety).

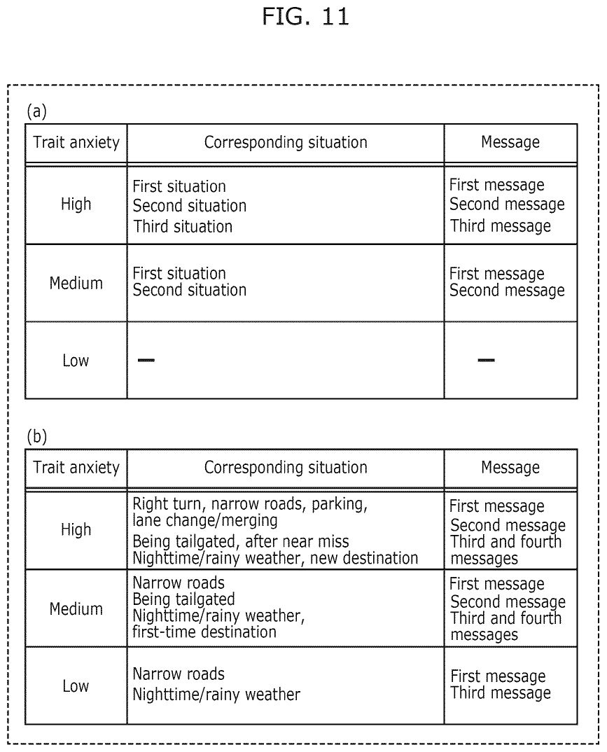

FIG. 11 is a diagram showing an example of messages to be presented to an occupant in information presentation system 1 according to a variation of the embodiment. (a) in FIG. 11 shows an example of messages according to the high and low trait anxiety for each type of situation, and (b) in FIG. 11 shows an example of messages according to the high and low trait anxiety for each specific situation.

In the example shown in (a) in FIG. 11, when the trait anxiety information indicates that the trait anxiety is “high”, determiner 141 determines to present a message to the occupant in any of the first, second, and third situations. In addition, in this example, when the trait anxiety information indicates that the trait anxiety is “medium”, determiner 141 determines to present the message to the occupant only in the first and second situations. In addition, in this example, when the trait anxiety information indicates that the trait anxiety is “low”, determiner 141 determines not to present a message to the occupant in any of the first, second, and third situations.

In the example shown in (b) in FIG. 11, when the trait anxiety information indicates that the trait anxiety is “high”, determiner 141 determines to present the first message to the occupant when the vehicle turns right, when the vehicle is traveling on a narrow road, when the vehicle is attempted to park, when the vehicle changes lane, or when the vehicle merges. In addition, in this example, when the trait anxiety information indicates that the trait anxiety is “high”, determiner 141 determines to present a second message to the occupant when the occupant is being tailgated or after a near miss. In addition, in this example, when the trait anxiety information indicates that the trait anxiety is high, determiner 141 determines to present the third or fourth message to the occupant when the vehicle is traveling at nighttime or in the rainy weather, or when the destination is the first time for the occupant.

In addition, in the example shown in (b) in FIG. 11, when the trait anxiety information indicates that the trait anxiety is “medium”, determiner 141 determines to present the first message to the occupant when the vehicle is traveling on a narrow road. In addition, in this example, when the trait anxiety information indicates that the trait anxiety is “medium”, determiner 141 determines to present a second message to the occupant when the occupant is being tailgated. In addition, in this example, when the trait anxiety information indicates that the trait anxiety is “medium”, determiner 141 determines to present the third message or fourth message to the occupant when the vehicle is travelling at nighttime or in the rainy weather, or when the destination is the first time for the occupant.

In addition, in the example shown in (b) in FIG. 11, when the trait anxiety information indicates that the trait anxiety is “low”, determiner 141 determines to present the first message to the occupant when the vehicle is travelling on a narrow road. In addition, in this example, when the trait anxiety information indicates that the trait anxiety is “low”, determiner 141 determines to present a third message to the occupant during the vehicle is travelling at nighttime or in the rainy weather.

In addition, for example, processing unit 14 may present an occupant with a message that the occupant desires regardless of the degree of the trait anxiety of the occupant. In this case, the occupant performs an operation in advance to select one or more situations to present a message by, for example, using an appropriate user interface, and select the type of message to be presented in each of the selected one or more situations. This causes processing unit 14 to present a message of the type desired by the occupant in each of the one or more situations desired by the occupant.

In the above embodiment, presenter 142 of processing unit 14 presents the message to the occupant by outputting the message as a voice from the speaker, but the present disclosure is not limited thereto. For example, presenter 142 may present the message to the occupant by causing the message to be displayed on a head-up display (HUD) provided in the vehicle. In addition, presenter 142 may present a message to the occupant both by outputting the voice from the speaker and by displaying the message on the HUD.

In the above embodiment, presenter 142 of processing unit 14 may change the type of voice output from the speaker according to the occupant. For example, presenter 142 may present a message to the occupant by outputting the voice of a male, a female, a child, an occupant's favorite actor, or an occupant's favorite character from the speaker, according to the occupant. In addition, for example, presenter 142 may present a message to the occupant by outputting the voice from the speaker in a dialect of Kansai dialect or the like according to the occupant.

In the above embodiment, information presentation system 1 may be implemented, for example, by an in-vehicle information (IVI) system, which is an in-vehicle information communication system using a car navigation system, a display, and a voice.

In addition, in the above embodiment, information presentation system 1 is implemented by in-vehicle devices, but the present disclosure is not limited thereto. For example, information presentation system 1 may be mounted on a server device separate from the vehicle, and may be configured to present a message to the occupant by performing wireless communication between the server device and the vehicle. In addition, information presentation system 1 may have a complex configuration in which part of it is mounted on a vehicle and the rest is mounted on a server device.

In addition, each unit included in information presentation system 1 according to the above embodiment is typically implemented as a large-scale integration (LSI) that is an integrated circuit. These may be individually formed into a single chip, or may be formed into a single chip so as to include some or all of them.

In addition, integration into an integrated circuit is not limited to LSI, and may be realized with a dedicated circuit or a general-purpose processor. A field programmable gate array (FPGA) that can be programmed after LSI manufacturing, or a reconfigurable processor that can reconfigure the connections and settings of circuit cells within the LSI may be utilized.

It should be noted that in the above embodiment, each component may include dedicated hardware or may be realized by executing a software program suitable for each component. Each component may be realized by a program executor such as a central processing unit (CPU) or a processor reading out and executing software programs recorded on a recording medium such as a hard disk or semiconductor memory.

In addition, all the numbers used above are illustrative to specifically explain the present disclosure, and the embodiment of the present disclosure are not limited to the illustrative numbers.

In addition, division of functional blocks in a block diagram is an example, and a plurality of functional blocks may be realized as one functional block, one functional block may be divided into a plurality of functional blocks, or some functions may be transferred to other functional blocks. In addition, functions of a plurality of functional blocks with similar functions may be processed in parallel or time division by a single hardware or software.

In addition, the order in which each step in the flow chart is performed is for illustrative purposes only to explain the present disclosure in detail, and may be in an order other than the above. In addition, some of the above steps may be performed simultaneously (in parallel) with other steps.

In addition, forms obtained by applying various modifications to the above embodiment conceived by a person skilled in the art or forms realized by arbitrarily combining the components and functions in the embodiment without departing from the spirit of the present disclosure are also included in this disclosure.

### CONCLUSION

As mentioned above, information presentation system 1 according to the first aspect includes first obtainer 11, second obtainer 12, and processing unit 14. First obtainer 11 obtains state anxiety information indicating state anxiety of an occupant of a vehicle. Second obtainer 12 obtains trait anxiety information indicating trait anxiety of the occupant. Processing unit 14 executes a process of presenting the occupant with a message for assisting the occupant in driving, based on the trait anxiety information, when the state anxiety information indicates that the state anxiety is occurring in the occupant.

According to this, by presenting the occupant with a message, there is an advantage that it is possible to assist the occupant from a psychological perspective taking the trait anxiety into account, and it is easier to reduce the anxiety that the occupant of the vehicle has when driving.

In addition, for example, in information presentation system 1 according to the second aspect, in the first aspect, processing unit 14 presents the message to the occupant when a value indicating the trait anxiety is higher than a threshold value, and does not present the message to the occupant when the value indicating the trait anxiety is lower than the threshold value.

According to this, the message is presented only to the occupant with relatively high trait anxiety, so that there is an advantage that it is easier to efficiently assist the occupant from a psychological perspective taking the trait anxiety into account.

In addition, for example, in information presentation system 1 according to the third aspect, in the first aspect, processing unit 14 determines a type of the message according to a level of a value indicating the trait anxiety.

According to this, the type of message presented to the occupant changes according to the degree of trait anxiety, so that there is an advantage that it is easier to efficiently assist not only the occupant with relatively high trait anxiety but also the occupant with relatively low trait anxiety from a psychological perspective taking the trait anxiety into account.

In addition, for example, information presentation system 1 according to the fourth aspect further includes third obtainer 13 that obtains situation information on a situation in which the occupant is placed, in any one of the first to third aspects. Processing unit 14 determines a type of the message according to the situation information.

According to this, there is an advantage that by presenting the occupant with a message to according to the situation in which the occupant is placed, it is easier to provide efficient assistance from a psychological perspective.

In addition, for example, in information presentation system 1 according to the fifth aspect, in the fourth aspect, when the situation information indicates that the situation is a first situation where there is no window of time for the occupant to cope with the situation, processing unit 14 presents the occupant with the message that is concise for coping with the first situation.

According to this, in situations where immediate judgment and immediate response is required, there is an advantage that by presenting the occupant with a concise message, it is easier for the occupant to cope with the situations, compared to when a message indicating a detailed coping method is presented to the occupant.

In addition, for example, in information presentation system 1 according to the sixth aspect, in the fourth or fifth aspect, when the situation information indicates that the situation is a second situation where there is a predetermined window of time for the occupant to cope with the situation, processing unit 14 presents the occupant with the message for diverting attention of the occupant from the state anxiety or the message for calming an anxious feeling.

According to this, in situations where anxiety is temporarily continued, there is an advantage that by presenting the occupant with the message for diverting attention of the occupant from the state anxiety or the message for calming an anxious feeling, it is easier for the occupant to concentrate on driving and reduce the anxiety of the occupant.

In addition, for example, in information presentation system 1 according to the seventh aspect, in any one of the fourth to sixth aspects, when the situation information indicates that the situation is a third situation where a window of time for the occupant to cope with the situation is longer than a predetermined time, processing unit 14 presents the occupant with the message according to a type of the third situation.

According to this, in situations where anxiety continues, there is an advantage that by presenting the occupant with a message according to the situations, it is easier to reduce the anxiety of the occupant.

In addition, for example, in information presentation system 1 according to the eighth aspect, in the seventh aspect, when the situation information indicates that the vehicle is in an environment that affects a field of view of the occupant, processing unit 14 presents the occupant with the message according to the environment.

According to this, in an environment where driving is difficult for the occupant, there is an advantage that by presenting the occupant with a message according to the environment, it is easier to increase the occupant's self-efficacy and reduce the anxiety of the occupant.

In addition, for example, in information presentation system 1 according to the ninth aspect, in the seventh or eighth aspect, when the situation information indicates that the occupant is heading to a destination that is new to the occupant, processing unit 14 presents the occupant with the message for providing information on the destination.

According to this, in an environment in which the occupant is worried about an unknown destination, there is an advantage that by providing information about the destination to the occupant, it is easier to reduce the expected anxiety of the occupant.

In addition, for example, in information presentation system 1 according to the tenth aspect, in any one of the fourth to sixth aspects, when the situation information indicates that the situation is a third situation where a window of time for the occupant to cope with the situation is longer than a predetermined time, processing unit 14 presents the occupant with the message that promotes reevaluation in cognitive change.

According to this, in situations where anxiety continues, presenting the occupant with a message that causes the occupant to change the way things or situations are perceived makes it easier for the occupant to reconsider the anxiety from a different perspective, so that there is an advantage that it is easy to reduce the anxiety of the occupant, and improvement of psychological skills to suppress the anxiety of the occupant can be expected.

In addition, for example, in information presentation system 1 according to the eleventh aspect, in any one of the first to tenth aspects, second obtainer 12 obtains the trait anxiety information based on a result of an answer to a question related to the trait anxiety for the occupant.

According to this, trait anxiety information is obtained based on the results of directly asking the occupant about trait anxiety, so that there is an advantage that it becomes easier to accurately grasp the degree of trait anxiety of the occupant. In addition, for example, in information presentation system 1 according to the twelfth aspect, in any one of the first to tenth aspects, second obtainer 12 obtains the trait anxiety information based on a degree of occurrence of the state anxiety of the occupant during driving.

According to this, trait anxiety information is obtained based on state anxiety actually experienced by the occupant during driving, so that there is an advantage that it becomes easier to accurately grasp the degree of trait anxiety of the occupant.

In addition, for example, in the information presentation method according to the thirteenth aspect, state anxiety information indicating state anxiety of an occupant of the vehicle is obtained. In the information presentation method, trait anxiety information indicating trait anxiety of the occupant is obtained. In the information presentation method, a process of presenting the occupant with a message for assisting the occupant in driving is executed based on the trait anxiety information, when the state anxiety information indicates that the state anxiety is occurring in the occupant.

According to this, it has the advantage that by presenting a message to the occupant, it is possible to assist the occupant from a psychological perspective, taking trait anxiety into account, and that it is easier to reduce the anxiety that the occupant of the vehicle has when driving.

In addition, for example, the program according to the fourteenth aspect causes one or more processors to execute the information presentation method according to the thirteenth aspect.

According to this, it has the advantage that by presenting a message to the occupant, it is possible to assist the occupant from a psychological perspective, taking trait anxiety into account, and that thit is easier to reduce the anxiety that the occupant of the vehicle has when driving.

While various embodiments have been described herein above, it is to be appreciated that various changes in form and detail may be made without departing from the spirit and scope of the present disclosure as presently or hereafter claimed.

### FURTHER INFORMATION ABOUT TECHNICAL BACKGROUND TO THIS APPLICATION

The disclosure of the following patent application including specification, drawings, and claims is incorporated herein by reference in their entirety: Japanese Patent Application No. 2024-082182 filed on May 20, 2024.

### INDUSTRIAL APPLICABILITY

The present disclosure can be used in systems for assisting the driving of a vehicle by an occupant in a mobile body, such as a vehicle or the like.

1. An information presentation system comprising:
memory; and
a processor connected to the memory,
wherein the processor:
obtains state anxiety information indicating state anxiety of an occupant of a vehicle;
obtains trait anxiety information indicating trait anxiety of the occupant; and
executes a process of presenting the occupant with a message for assisting the occupant in driving, based on the trait anxiety information, when the state anxiety information indicates that the state anxiety is occurring in the occupant.

2. The information presentation system according to claim 1,
wherein the processor presents the message to the occupant when a value indicating the trait anxiety is higher than a threshold value, and does not present the message to the occupant when the value indicating the trait anxiety is lower than the threshold value.

3. The information presentation system according to claim 1,
wherein the processor determines a type of the message according to a level of a value indicating the trait anxiety.

4. The information presentation system according to claim 1,
wherein the processor further obtains situation information on a situation in which the occupant is placed, and determines a type of the message according to the situation information.

5. The information presentation system according to claim 4,
wherein when the situation information indicates that the situation is a first situation where there is no window of time for the occupant to cope with the situation, the processor presents the occupant with the message that is concise for coping with the first situation.

6. The information presentation system according to claim 4,
wherein when the situation information indicates that the situation is a second situation where there is a predetermined window of time for the occupant to cope with the situation, the processor presents the occupant with the message for diverting attention of the occupant from the state anxiety or the message for calming an anxious feeling.

7. The information presentation system according to claim 4,
wherein when the situation information indicates that the situation is a third situation where a window of time for the occupant to cope with the situation is longer than a predetermined time, the processor presents the occupant with the message according to a type of the third situation.

8. The information presentation system according to claim 7,
wherein when the situation information indicates that the vehicle is in an environment that affects a field of view of the occupant, the processor presents the occupant with the message according to the environment.

9. The information presentation system according to claim 7,
wherein when the situation information indicates that the occupant is heading to a destination that is new to the occupant, the processor presents the occupant with the message for providing information on the destination.

10. The information presentation system according to claim 4,
wherein when the situation information indicates that the situation is a third situation where a window of time for the occupant to cope with the situation is longer than a predetermined time, the processor presents the occupant with the message that promotes reevaluation in cognitive change.

11. The information presentation system according to claim 1,
wherein the processor obtains the trait anxiety information based on a result of an answer to a question related to the trait anxiety for the occupant.

12. The information presentation system according to claim 1,
wherein the processor obtains the trait anxiety information based on a degree of occurrence of the state anxiety of the occupant during driving.

13. An information presentation method comprising:
obtaining state anxiety information indicating state anxiety of an occupant of a vehicle;
obtaining trait anxiety information indicating trait anxiety of the occupant; and
executing a process of presenting the occupant with a message for assisting the occupant in driving, based on the trait anxiety information, when the state anxiety information indicates that the state anxiety is occurring in the occupant.
